(19) United States
(12) Patent Application Publication
(10) Pub. No.: US 2019/0015252 A1
(43) Pub. Date: Jan. 17, 2019

(54) CATARACT EXTRACTION METHOD AND INSTRUMENTATION

(71) Applicants: Jonathan Lake, Brasillia (BR); Fabio Henrique Cacho Casanova, Recife (BR); Francisco Seixas Soares, Sao Paulo (BR); Alfredo Tranjan, Sao Paulo (BR)

(72) Inventors: Jonathan Lake, Brasillia (BR); Fabio Henrique Cacho Casanova, Recife (BR); Francisco Seixas Soares, Sao Paulo (BR); Alfredo Tranjan, Sao Paulo (BR)

(21) Appl. No.: 15/651,214

(22) Filed: Jul. 17, 2017

(51) Int. Cl. A61F 9/007; A61B 17/32; A61F 9/008

(52) U.S. Cl. CPC A61F 9/00745; A61B 17/320068; A61B 2217/007; A61F 2009/00887; A61B 2217/005; A61F 9/008

(57) ABSTRACT

A tip for a phacoemulsification handpiece, including a tubular structure having an elongate portion with generally parallel sides and a flared end portion extending outwardly from the elongate portion and presenting a mouth that is larger in diameter than a lumen diameter of the elongate portion. The tip may include a plurality of internally extending structures coupled to an interior of the tubular structure. A method of surgically removing a crystalline lens of an eye, including evaluating the crystalline lens to determine the level of nuclear sclerosis, sectioning at least a central portion of the crystalline lens into a plurality of lens fragments by application of femtosecond laser energy and aspirating the lens fragments from the eye using a phaco-aspiration tip coupled to a source of suction. The method may include application of pulsed vacuum to aspirate lens fragments.

### TECHNICAL FIELD

Embodiments of the invention generally relate to the field of cataract surgery. More particularly, embodiments of the invention relate to the extraction of fragments of the crystalline lens from within the lens capsule.

### BACKGROUND

The human eye includes a lens system formed in part by the cornea and the crystalline lens that focuses light on the retina.

In youth, the crystalline lens is a lens that is capable of adjustable focus according to distance. In youth the crystalline lens is highly flexible and the ability to accommodate or to focus at near is highly flexible as well. The crystalline lens changes by becoming more rigid and opaque throughout human life due to aging changes. Other diseases or environmental factors may lead to lens opacification. Lens opacification and density may present in several grades, varying from mild to severe. Lens opacification is commonly known as cataract. The term cataract will be used to refer to any kind of lens opacification or dysfunction for the purposes of this application.

Cataracts are a leading cause of vision loss and sometimes blindness worldwide. The interference with the passage of light through the natural crystalline lens causes images to become cloudy and distorted, thereby diminishing visual acuity. Severe diminishment of visual acuity from cataracts can lead to an increase in auto accidents, falls, and other social problems. Generally, in the developed world, cataract surgery is performed well prior to severe reduction in vision as a consequence of the cataract.

The crystalline lens has been compared to an onion in structure, having multiple layers of cells surrounding the lens nucleus which is itself made up of cells. The lens nucleus is generally denser than the surrounding lens cortex. In particular, in nuclear sclerotic cataracts the lens nucleus becomes more dense and hard as the cataract develops. This can make fragmentation of the nucleus to facilitate lens removal more difficult.

For the past several decades modern cataract surgery has commonly been performed by so-called extracapsular cataract extraction utilizing the technique of phacoemulsification. This procedure uses a phacoemulsification device to break the crystalline lens including the cataract into small pieces by the application of ultrasonic energy. Ultrasonic energy may damage ocular tissue however, if used in controlled small amounts, whatever damage that may be done does not result in loss of visual function. The same phacoemulsification device uses irrigation and aspiration to remove the fragments during the procedure.

In this known technique, the crystalline lens is removed while leaving at least part of the lens capsule that surrounds the crystalline lens intact. An opening is made in the anterior lens capsule to provide access to the lens within. This opening is sometimes referred to as a capsulorhexis and can be created manually or by the application of laser energy. Phacoemulsification is performed with a handheld instrument coupled to a console. The handheld instrument includes an ultrasonically vibrating tip, and aspiration tube and a conduit through which a liquid, such as a balanced salt solution, is supplied to replace fluid that is removed from the eye during aspiration.

More recently another approach to cataract surgery has been developed in which femtosecond laser pulses are applied to cut or section the crystalline lens into smaller pieces to facilitate removal of the lens. Femtosecond laser can be utilized to create incisions within the structure of the crystalline lens. Femtosecond lasers have been used to fragment the cataract lens prior to phacoemulsification. So far, this has been used to reduce ultrasonic energy applied during phacoemulsification.

Sectioning of the lens by application of femtosecond laser is expected to be a significant benefit to cataract surgery as it reduces the amount of ultrasound energy that is applied to the eye. It is known to those skilled in the art that ultrasound application may damage structures within the eye. In particular, the corneal endothelium may be negatively affected by excess ultrasound energy being applied to the eye during phacoemulsification.

Accordingly, there is still room in the eye surgical arts to improve cataract surgery techniques.

### SUMMARY

Embodiments of the invention disclosed herein solve many of the above discussed problems further related to cataract surgery. Example embodiments of the invention are expected to minimize or even to eliminate ultrasound application during aspiration of the human lens cortex and nucleus during cataract surgery. Example embodiments of the invention include techniques for the application of femtosecond laser as well as methods and devices related to lens fragment aspiration.

According to an example embodiment of the invention, utilized when the lens nucleus is “softer,” femtosecond laser pulses are applied to accomplish fragmentation of at least a central portion of the crystalline lens. “Softer” is defined in the context of this application as a lens is judged to have a 2+ or less nuclear sclerosis. The fragmentation may be cuboidal, for example. That is, roughly cube shaped. The central portion of the crystalline lens may include at least a portion of the lens nucleus. In alternate embodiments, femtosecond energy is applied to the lens structure to section all or substantially all of the crystalline lens. In this context, “substantially all” means 90% or more of the volume of the crystalline lens. According to an example embodiment of the invention, a portion of the lens is sectioned into cuboids approximately 300 μm on each side. In accordance with this embodiment of the invention “approximately” means 300 μm plus or minus 50 μm. It is expected that this approach will be effective with lenses presenting with up to grade 2 nuclear sclerosis. Other shapes are contemplated as well and neither the cuboidal nature of the sectioned portions nor the example dimensions of 300 μm should be considered to be limiting.

According to another example embodiment of the invention, which is intended to be used when the lens nucleus is “harder” as defined by the lens presenting grade 3+ or greater nuclear sclerosis, a further laser pulse or multiple further laser pulses are applied at or near a center of at least some of the cuboidal sections created by the application of femtosecond laser pulses. The application of a further laser pulse or pulses creates a plasma bubble within the cuboidal section to further soften or assist in the deconstruction of the cuboidal section. The further laser pulses may be applied prior to or following the creation of the cuboidal sections. Further, sectioning into cuboids and the application of the further laser pulse or pulses within cuboidal sections may be accomplished alternately.

It is generally desirable to apply femtosecond laser pulses starting at a location in the tissue farthest from the laser source. This prevents the plasma bubbles from earlier pulse applications from interfering with the application of later pulses. Thus, a layer or row of cuboidal sections may be created followed by application of the central laser pulse or pulses. Alternately, a single cuboidal section may be created followed by application of the central laser pulse or pulses to that cuboidal section. In further alternate embodiments, central laser pulse or pulses may be applied followed by creation of a cuboidal section around each central laser pulse or pulses.

According to one example embodiment, at least a central portion of lens is sectioned into cubes of approximately 300 μm or another selected size. After, before or during cuboidal fragmentation, a further central femtosecond laser pulse is applied proximate the center of at least some of the 300 μm cuboids to further fragment, disrupt or soften the 300 μm cubes. It is expected that the formation of a plasma bubble at or near the center of the 300 μm cubes will cause the treated cubes to further fragment, disrupt or at least, to be softened and thus facilitate later aspiration.

Another example embodiment of the invention includes a phaco-aspiration tip having a central lumen and a frustoconical or funnel-shaped aspiration intake. According to an example embodiment of the invention, the aspiration tip has a tubular structure defining a lumen of approximately 900 μm in diameter. In this context the term “approximately” means a lumen having a diameter of 900 μm plus or minus 100 μm. According to an example embodiment, the intake opening of the phaco-aspiration tip has an inside diameter of approximately 1200 μm. In this context “approximately” should be understood to mean 1200 μm plus or minus 100 μm. Other sizes of the lumen diameter and the intake opening diameter are contemplated as part of the invention.

According to another example embodiment of the invention, the phaco-aspiration tip presents internally extending structures such as, spikes, blades or other structures located proximate the junction between the frustoconical portion and the cylindrical portion or at another location within the lumen. For example, the internally extending structures can be four in number located at approximately 90° intervals about a circumference of the lumen. A larger or smaller number of internally extending structures may be utilized. It is contemplated that the internally extending structures may be spaced evenly or unevenly about the internal circumference of the phaco-aspiration tip. According to an example embodiment of the invention, the internally extending structures may extend inwardly from 100 to 400 μm. It is expected that features of this tip design will assist in apprehension and disassembly of cuboidal fragments that are to be aspirated and removed from the eye.

According to another example embodiment of the invention, a pulsed vacuum is applied via the phaco-aspiration tip to facilitate deconstruction and aspiration of the cuboidal fragments or cuboids. Vacuum is applied in a pulsed fashion to aspirate lens fragments and to assist in their disruption are disassembly. For example, vacuum pressure may be pulsed between vacuum settings of 0 to 100 mmHg. In a further example, vacuum pressure may be pulsed between vacuum of 100 to 500 mmHg. According to an example embodiment of the invention, micro vacuum surges are generated and expected to deconstruct and aspirate the nuclear cuboids without losing prehension or grasping of the nucleus fragments. Pulsed vacuum may be utilized in combination with the tip design having a funnel shaped opening and internally extending structures to facilitate aspiration and passage of the cuboids.

Example embodiments of the invention facilitate the minimization or elimination of application of ultrasound energy to the eye. According to embodiments of the invention, it is expected that any ultrasound energy applied will be minimal and independent of surgeon decision-making during the procedure. This means that the surgeon will decide in advance a maximum amount of ultrasonic energy to be applied and set the system to apply no more than this amount of ultrasonic energy. Ultrasound, if applied, is thus activated automatically and according to a predetermined threshold determined by the surgeon. It is expected that with use of the embodiments of the invention it should only be necessary to apply ultrasound, if at all, in harder cataract nuclei demonstrating approximately grade 3 nuclear sclerosis or above.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a schematic cross sectional depiction of a crystalline lens which has been divided by femtosecond laser application into 300 μm cuboids according to an example embodiment of the invention;

FIG. 2 is a schematic cross sectional depiction of a crystalline lens which has been divided by femtosecond laser application into 300 μm cuboids and to which a central femtosecond pulse application has been applied to facilitate lens aspiration according to an example embodiment of the invention;

FIG. 3 is a side schematic cross sectional view of a phaco aspiration tip according to an example embodiment of the invention;

FIG. 4 is a front sectional view of a lumen of a frustoconical portion of the phaco aspiration tip including internally directed spikes according to an example embodiment of the invention;

FIG. 5 is a flowchart depicting a method according to an example embodiment of the invention;

FIG. 6 is a schematic cross sectional depiction of a crystalline lens depicting a crystalline lens including the nucleus and cortex.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

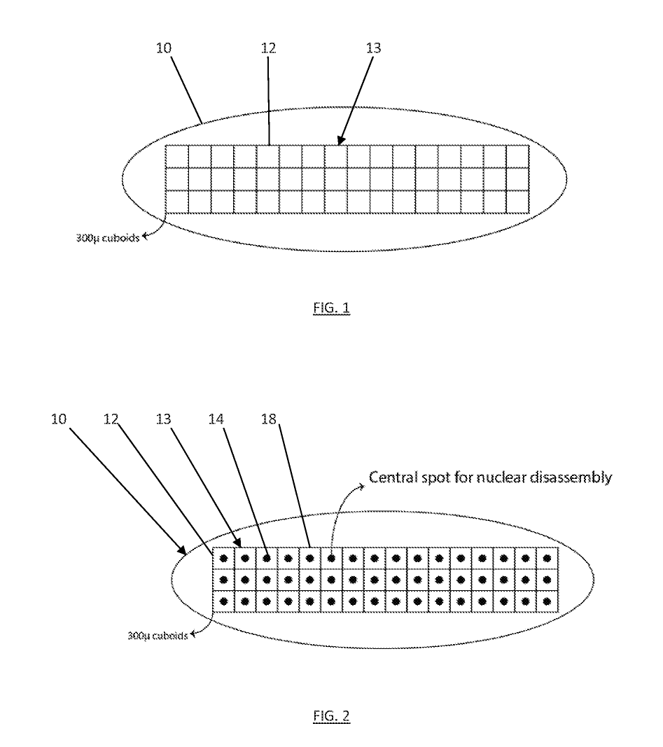

Referring to FIGS. 1-6, according to an example embodiment of the invention crystalline lens 10 is sectioned by femtosecond laser energy into a plurality of cuboids 12. According to an example embodiment depicted in FIG. 1, cuboids 12 are approximately 300 μm on each side. While the volume occupied by cuboids 12 is depicted as occupying a particular portion of the schematically depicted crystalline lens 10 this should not be considered limiting. Volume 13 occupied by cuboids 12 can be greater or lesser than that schematically depicted. Nor should the 300 μm dimension be considered limiting as the invention contemplates other sizes. Cuboids 12 need not be aligned as in the schematic depiction. The alignment depicted is purely an example.

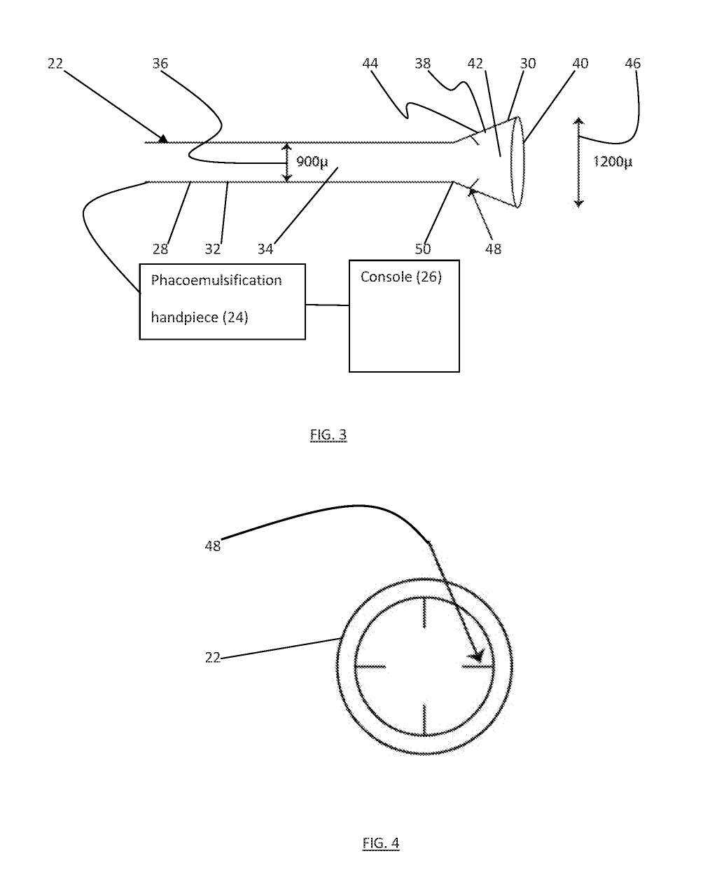

Referring particularly to FIG. 2, according to another example embodiment of the invention each cuboid 12 may be occupied and further treated by at least one plasma bubble 14. Pulsed femtosecond laser energy may be applied to a central portion of cuboid 12 to create centrally located plasma bubble 14 to support disruption and disassembly of cuboids 12 occupying a treated cuboidal volume 13 in FIG. 2.

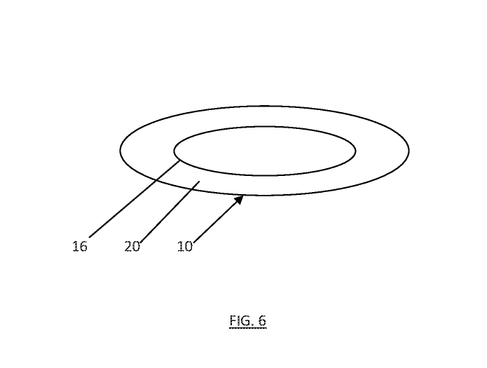

As is known to those skilled in the art and depicted schematically in FIG. 6, lens nucleus 16 is generally centrally located in crystalline lens 10. Lens nucleus 16 is denser than lens cortex 20, particularly in crystalline lens 10 that is affected by a nuclear sclerotic cataract. Treated cuboidal portion 18 of crystalline lens 10, for the purposes of this application generally includes volume 13 of crystalline lens 10 that is sectioned by femtosecond laser and which contains cuboids 12.

Referring now to FIG. 3, according to an example embodiment, the invention includes a phaco-aspiration tip 22. Phaco-aspiration tip 22 is intended to be coupled to generally conventional phacoemulsification handpiece 24. Phacoemulsification handpiece 24 is in turn coupled to console 26 which supplies vacuum for aspiration, fluid for fluid replacement and electrical power for ultrasonic energy application if needed. Console 26 is generally conventional, and because it is known to those skilled in the art, will not be further described here.

Phaco-aspiration tip 22 generally includes tubular portion 28 and end portion 30. Tubular portion 28 and end portion 30 may be formed from rigid materials such as Nitinol or stainless steel or other rigid materials and may be formed, for example, as a unitary structure.

Tubular portion 28 generally presents elongate tube 32 defining lumen 34. According to the depicted example embodiment, elongate tube 32 has inner diameter 36 of approximately 900 μm. In this context, for the purposes of this application “approximately” means 900 μm plus or minus 100 μm.

End portion 30 generally presents flared portion 38 defining mouth 40, flared lumen 42 and narrow end 44. Flared portion 38 may also be bell-shaped or otherwise curved rather than straight sided. Mouth 40 has an inner diameter 46 greater than inner diameter 36. Narrow end 44 meets and is coupled to tubular portion 28 and has inner diameter 36.

End portion 30 further presents internally extending structures 48. Internally extending structures 48 may take the form of spikes or blades. Internally extending structures 48 may, in the depicted example, include four internally extending structures 48 located at approximately 90° intervals around an internal circumference of flared lumen 42. A greater or lesser number of internally extending structures 48 may be utilized.

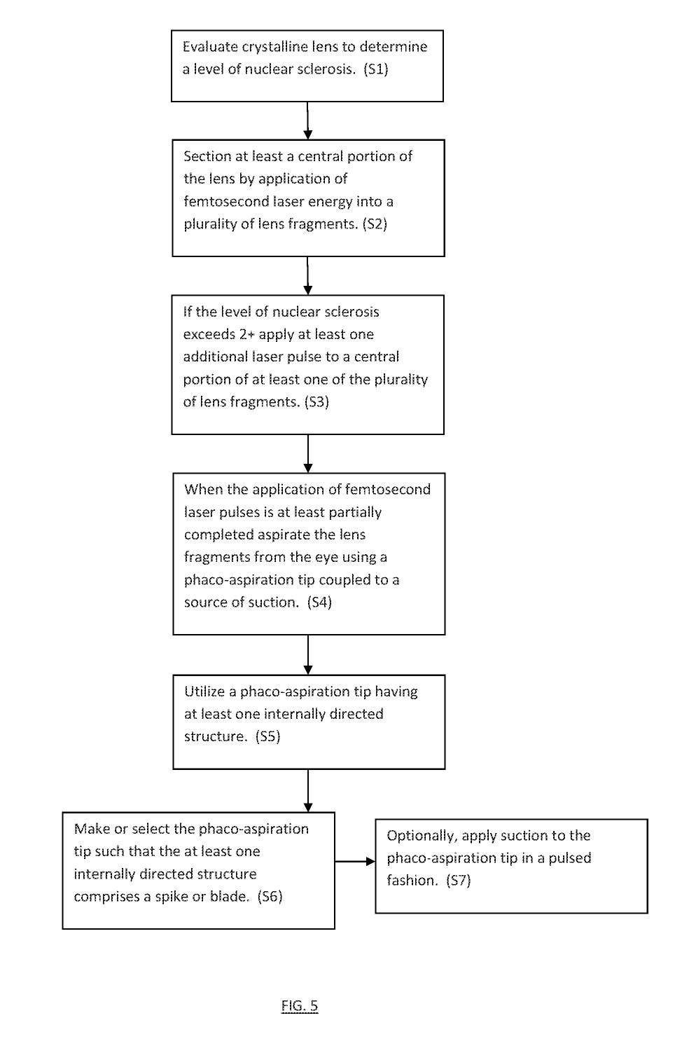

Referring now to FIG. 4, internally extending structures 48 may be spaced uniformly or non-uniformly within flared lumen 42. According to other example embodiments, internally extending structures 48 may be located within elongate tube 32 or at junction 50 between elongate tube 32 and frustoconical portion 38. Internally extending structures 48 may be formed of the same material as tubular portion 28 or may be formed of a different material coupled to put tubular portion 28.

Internally extending structures 48 may extend inwardly normal to a longitudinal axis of phaco-aspiration tip 22, may be tilted toward mouth 40 as depicted in FIG. 3 or may be tilted away from mouth 40. Internally extending structures 48 may vary in length from 100 to 400 μm.

Other embodiments of the invention include a method that includes evaluating a crystalline lens to determine a level of nuclear sclerosis S1. The method further includes sectioning at least a central portion of a crystalline lens into a plurality of lens fragments or cuboids 12 by application of femtosecond laser energy S2. If the level of nuclear sclerosis exceeds a preselected value, for example, 2+ embodiments of the method includes applying at least one additional laser pulse to a central portion of at least one of the plurality of lens fragments to create plasma bubble 14 thus creating a treated cuboidal portion 18 S3. When the application of femtosecond laser pulses is at least partially completed, the method may include, aspirating lens fragments or cuboids 12 from the eye using a phaco-aspiration tip 22 coupled to a source of suction such as console 26 S4. According to a further example embodiment, phaco—aspiration tip is utilized including at least one internally directed structure S5. According to another example embodiment of the invention, the method includes making or selecting the phaco-aspiration tip such that the at least one internally directed structure comprises a spike or blade S6. The method may optionally include applying suction to the phaco-aspiration tip in a pulsed fashion S7.

In operation, femtosecond laser energy is applied to crystalline lens 10 to section at least a partial volume 13 of crystalline lens 10 into cuboids 12. Optionally, in the case of crystalline lens 10 having, for example, 3+ and above nuclear sclerosis, femtosecond laser energy is further applied to crystalline lens 10 to further create plasma bubble 14 within cuboids 12, for example, at or near a center of cuboids 12. The application of femtosecond energy to create plasma bubble 14 creates treated cuboidal portion 18. Volume 14 may include at least a portion of lens nucleus 18. According to an example embodiment volume 14 encompasses all of lens nucleus 18. The application of femtosecond laser energy to create plasma bubble 14 is expected to be useful to facilitate aspiration in the case of a lens nucleus demonstrating, for example, 3+ nuclear sclerosis or greater. According to different embodiments of the invention, volume 13 may encompass a portion of crystalline lens 10 or substantially all of crystalline lens 10.

Following sectioning by application of femtosecond laser energy phaco-aspiration tip 22 is used to aspirate cuboids 12 or treated cuboidal portions 18. It is expected that aspiration can be accomplished with a minimal application of ultrasonic energy. It is expected that with the use of phaco-aspiration tip 22 it will be possible to aspirate the material of crystalline lens 10 completely without the application of ultrasonic energy.

Phaco-aspiration tip 22 is manipulated via phacoemulsification handpiece 24 to place mouth 40 of frustoconical portion 38 in proximity of cuboids 12. This is accomplished through a corneal or scleral incision as is known to those skilled in the art. It is expected that with the application of steady or pulsed vacuum cuboids 12 or treated cuboidal portion 18 of lens nucleus 16 and lens cortex 20 will be aspirated and removed from the eye.

When brought into proximity or contact with mouth 40 and flared lumen 42 of phaco-aspiration tip 22, cuboids 12 or treated cuboidal portions 18 are drawn into flared lumen 42 by application of suction. Cuboids 12 or treated cuboidal portions 18 encounter and are engaged by internally extending structures 48. Internally extending structures 48 assist in the prehension and retention of cuboids 12 or treated cuboidal portions 18 within flared lumen 42 which are then aspirated through lumen 34 of elongate tube 32. Internally extending structures 48 may assist in deconstruction of cuboids 12 thus assisting the aspiration process. Once cuboids 12 or treated cuboidal portions 18 pass junction 50 they are overcome by fluid flow and are expected to be removed permanently from the eye.

In an alternative embodiment, pulsed suction is applied via lumen 34. In this embodiment, micro vacuum surges of suction assist in drawing cuboids 12 or treated cuboidal portions 18 through flared lumen 42 into lumen 34 and onward to console 26.

After lens nucleus 16 and lens cortex 20 of crystalline lens 10 are removed, an intraocular lens implant may be placed in the lens capsule according to techniques known to those skilled in the art.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A tip for a phacoemulsification handpiece, comprising:
a tubular structure including an elongate portion having generally parallel sides and a flared end portion extending outwardly from the elongate portion and presenting a mouth that is larger in diameter than a lumen diameter of the elongate portion;
a plurality of internally extending structures coupled to an interior of the tubular structure;
wherein the tubular structure is coupled to the phacoemulsification handpiece which in turn is coupled to a source of suction by which fragments of a crystalline lens may be drawn into the lumen of the tubular structure.

2. The tip for a phacoemulsification handpiece as claimed in claim 1, wherein the internally extending structures comprise spikes or blades.

3. The tip for a phacoemulsification handpiece as claimed in claim 1, wherein the flared end portion is frustoconical in shape.

4. The tip for a phacoemulsification handpiece as claimed in claim 1, wherein the mouth defines an inner diameter of approximately 1200 μm.

5. The tip for a phacoemulsification handpiece as claimed in claim 1, wherein the internally extending structures are located within the flared end portion.

6. The tip for a phacoemulsification handpiece as claimed in claim 1, wherein the internally extending structures extend internally a distance between 100 and 400 μm

7. The tip for a phacoemulsification handpiece as claimed in claim 1, wherein the internally extending structures are located proximate a junction between the flared end portion and the elongate portion having generally parallel sides.

8. The tip for a phacoemulsification handpiece as claimed in claim 1, wherein the internally extending structures are located within the elongate portion having generally parallel sides.

9. The tip for a phacoemulsification handpiece as claimed in claim 1, wherein the source of vacuum is structured to supply vacuum in a pulsed fashion.

10. The tip for a phacoemulsification handpiece as claimed in claim 1, wherein the tubular structure defines an inner diameter of approximately 900 μm.

11. A method of surgically removing a crystalline lens of an eye, the method comprising:
evaluating the crystalline lens to determine a level of nuclear sclerosis;
sectioning at least a central portion of the crystalline lens into a plurality of lens fragments by application of femtosecond laser energy;
aspirating the lens fragments from the eye using a phaco-aspiration tip coupled to a source of suction;
selecting or making the phaco-aspiration tip to include an elongate tube defining a flared end portion and a parallel side tubular portion.

12. The method as claimed in claim 11, further comprising engaging internally extending structures of the phaco-aspiration tip with the lens fragments.

13. The method as claimed in claim 11, further comprising applying no ultrasound energy to the eye during aspiration.

14. The method as claimed in claim 11, further comprising applying aspiration suction in a pulsed fashion.

15. The method as claimed in claim 11, further comprising sectioning the crystalline lens so that the lens fragments comprise cuboids.

16. The method as claimed in claim 15, further comprising making the cuboids to have it dimension of approximately 300 μm.

17. The method as claimed in claim 11, further comprising applying at least one further femtosecond laser pulse centrally within at least some of the lens fragments when the level of nuclear sclerosis exceeds a preselected value.

18. The method as claimed in claim 17, further comprising applying the at least one further femtosecond laser pulse when the preselected value for the level of nuclear sclerosis exceeds 2+.

19. The method as claimed in claim 11, further comprising applying additional femtosecond laser energy to at least some of the lens fragments thereby facilitating disruption of the lens fragments and aspiration of the lens fragments to which additional femtosecond laser energy is applied.

20. The method as claimed in claim 11, further comprising sectioning at least a portion of a lens nucleus by the application of femtosecond laser energy.

21. The method as claimed in claim 11, further comprising sectioning substantially an entirety of the crystalline lens by the application of femtosecond laser energy.
